(19) United States
(12) Patent Application Publication
(10) Pub. No.: US 2009/0221951 A1
(43) Pub. Date: Sep. 3, 2009

(54) IONTOPHORESIS DEVICE AND ELECTRIC POWER SOURCE FOR AN IONTOPHORESIS DEVICE

(76) Inventors: Mizuo Nakayama, Tokyo (JP); Takehiko Matsumura, Tokyo (JP); Hidero Akiyama, Tokyo (JP); Akihiko Matsumura, Tokyo (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 12/066,362

(22) PCT Filed: Sep. 13, 2006
(86) PCT No.: PCT/JP2006/318174

§ 371 Date: Apr. 10, 2009

(30) Foreign Application Priority Data
Sep. 14, 2005 (JP) 2005-267588

(51) Int. Cl. A61N 1/30

(52) U.S. Cl. 604/20

(57) ABSTRACT

A drug may be administered in accordance with a daily living pattern of a patient and/or a circadian rhythm of the drug.

An iontophoresis device includes a working electrode assembly and a non-working electrode assembly for administering a drug by iontophoresis and an electric power source connected to the working electrode assembly and the non-working electrode assembly. Current control circuitry programmably controls a current that flows to each of the electrode assemblies according to a set pattern, and the drug is released based at least in part on the current.

### CROSS-REFERENCE TO RELATED APPLICATIONS

This application is the U.S. national stage of international application no. PCT/JP2006/318174, filed Sep. 13, 2006, which claims benefit from Japanese application no. 2005-267588, filed Sep. 14, 2005, which applications are hereby incorporated by reference in their entirety.

### BACKGROUND

1. Technical Field

The present description relates to an iontophoresis device for administering a drug to an organism and an electric power source for the iontophoresis device.

2. Description of the Related Art

Iontophoresis is one method of permeating a drug into an organism through a biological interface, such as the skin or mucosa. Iontophoresis devices typically include a working electrode assembly having a drug solution holding portion holding a drug solution and a non-working electrode assembly as a counter electrode for the working electrode assembly. A voltage having the same polarity as that of a drug (e.g., a drug ion) in the drug solution holding portion is applied to the working electrode assembly as it is brought into close contact with a biological interface to electrically drive the drug into an organism via the biological interface.

A patch-type structure is one possible structure for each of the working electrode assembly and the non-working electrode assembly.

The term “patch” conventionally refers to a cloth-like section having adhesive on one surface that may also include a substance such as a drug or an antigen. The patch may have some thickness and may have no adhesiveness in some embodiments.

As described in, for example, JP 2002-532540 A, a nicotine patch containing nicotine as an antismoking auxiliary agent is one example of such a patch. In addition, as described in JP 2005-510488 A, some transdermal absorption patches are used to deliver local anesthetics, e.g., using morphine hydrochloride.

Drug tapes have also been developed. A film of the drug tape is typically configured such that the amount of drug released reaches its peak at some point in time (e.g., 6 hours) after the tape is placed against the biological interface.

Conventionally, the amount of drug administered by such methods cannot be finely controlled. For example, a drug cannot be easily administered in accordance with the daily patterns of a patient, an onset time (for example, dawn, in the case of an asthmatic attack), a circadian rhythm, or otherwise.

Oral delivery may apply an excessive load to a patient's stomach. Meanwhile, if one attempts to manage the amount of drug administered by removing a patch-type iontophoresis device after a predetermined time period, it is relatively easy to forget this manual task and the amount of drug administered may exceed an allowable value.

### BRIEF SUMMARY

One object of the embodiments described herein is to provide an iontophoresis device capable of controlling the amount and timing of drug administration. This may be used to take into consideration the daily patterns of a patient and the circadian rhythm of the drug, for example.

In one embodiment, an iontophoresis device may include: a working electrode assembly and a non-working electrode assembly for administering a drug by iontophoresis; an electric power source connected to the working electrode assembly and the non-working electrode assembly; and current control circuitry coupled to the electric power source and operable to programmably control a current that flows to each of the electrode assemblies according to a set pattern; wherein the drug is released based at least in part on the current.

In one embodiment, the set pattern of the current control circuitry is rewritable from outside the iontophoresis device without contact.

In another embodiment, the working electrode assembly and the non-working electrode assembly are each of a patch-type.

In yet another embodiment, the working electrode assembly includes: a working electrode connected to the electric power source with a same polarity as that of a charged ion of the drug; an electrolyte solution holding portion holding an electrolyte solution, the electrolyte solution holding portion adjacent a front surface of the working electrode; a second ion exchange membrane permitting passage of ions having a polarity opposite to that of the charged ion of the drug, the second ion exchange membrane adjacent a front surface of the electrolyte solution holding portion; a drug solution holding portion holding the drug, the drug solution holding portion adjacent a front surface of the second ion exchange membrane; and a first ion exchange membrane permitting passage of ions having the same polarity as that of the charged ion of the drug, the first ion exchange membrane adjacent a front surface of the drug solution holding portion. The non-working electrode assembly may also include: the non-working electrode connected to the electric power source having the polarity opposite to that of the charged ion of the drug; a second electrolyte solution holding portion holding a second electrolyte solution, the second electrolyte solution holding portion adjacent a front surface of the non-working electrode; a third ion exchange membrane permitting passage of ions having the same polarity as that of the charged ion of the drug, the third ion exchange membrane adjacent a front surface of the second electrolyte solution holding portion; a third electrolyte solution holding portion holding a third electrolyte solution, the third electrolyte solution holding portion adjacent a front surface of the third ion exchange membrane; and a fourth ion exchange membrane permitting passage of ions having the polarity opposite to that of the charged ion of the drug, the fourth ion exchange membrane adjacent a front surface of the third electrolyte solution holding portion.

In yet another embodiment, an electric power source device for an iontophoresis device including a working electrode assembly and a non-working electrode assembly for administering a drug by iontophoresis is disclosed. The electric power source device may include: a battery; current control circuitry operable to programmably control a current from the battery to each of the electrode assemblies according to a set pattern; and an antenna operable to receive a signal used to rewrite the set pattern of the current control circuitry without contact.

In another embodiment, the current control circuitry may be disposed radially outside the battery and side by side next to the battery.

In yet another embodiment, the battery is disposed off-center from the working electrode assembly, and the current control circuitry is disposed next to the battery on a side of the battery closer to a center of the working electrode assembly.

In still another embodiment, the current control circuitry is disposed on top of the battery.

A current amount and a discharge duration may be controlled by using a program to implement a pattern determined by a doctor. The amount of drug administered by an iontophoresis device is proportional to the current, so the amount and timing of drug administered may thereby be controlled. As a result, the drug can be administered in accordance with, for example, the living pattern of a patient and/or a circadian rhythm.

In addition, in certain embodiments, an iontophoresis device may be erroneously stuck to a skin for a long time period, while the amount of drug administered is limited to an allowable value or less, by causing the current to go to zero after a predetermined discharge duration (i.e., administration time).

### BRIEF DESCRIPTION OF THE SEVERAL VIEWS OF THE DRAWINGS

In the drawings, identical reference numbers identify similar elements. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a plan view of an iontophoresis device, according to one illustrated embodiment.

FIG. 2 is an enlarged cross-sectional view of the iontophoresis device of FIG. 1 taken along the line II-II.

FIG. 3 is an enlarged cross-sectional view of the iontophoresis device of FIG. 1 taken along the line III-III.

FIG. 4 is a circuit diagram representing an electrical system of the iontophoresis device of FIG. 1.

FIG. 5 is a circuit diagram of current control circuitry of the iontophoresis device of FIG. 1, according to one illustrated embodiment.

FIG. 6 illustrates a set pattern for a current that flow to electrode assemblies of the iontophoresis device of FIG. 1.

FIG. 7 is a plan view of an electric power source and current control circuitry according to another illustrated embodiment.

FIG. 8 is a cross-sectional view of an electric power source and current control circuitry, according to another illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices and power sources for iontophoresis devices have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

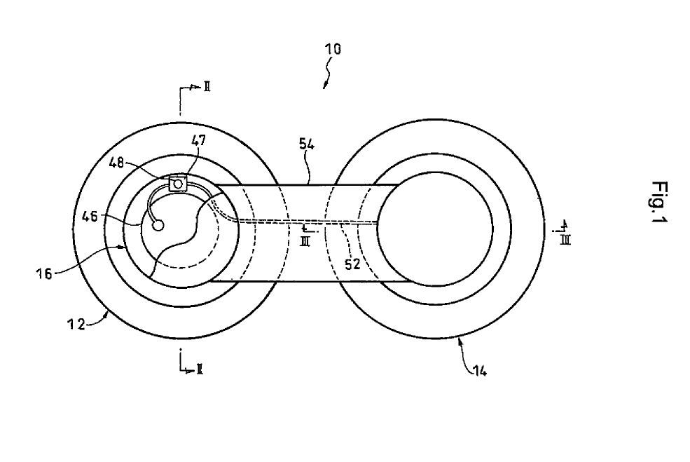

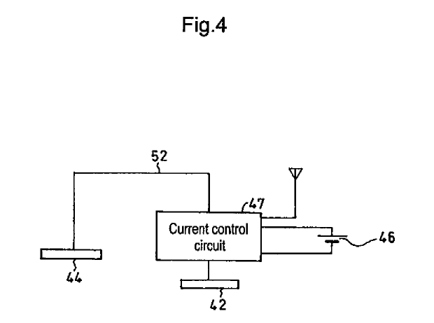

In one embodiment, as shown in FIGS. 1 and 4, an iontophoresis device 10 comprises a working electrode assembly 12 and a non-working electrode assembly 14 for administering a drug (e.g., an ionic drug), and a DC electric power source 16 connected to the electrode assemblies 12, 14 with opposite polarities.

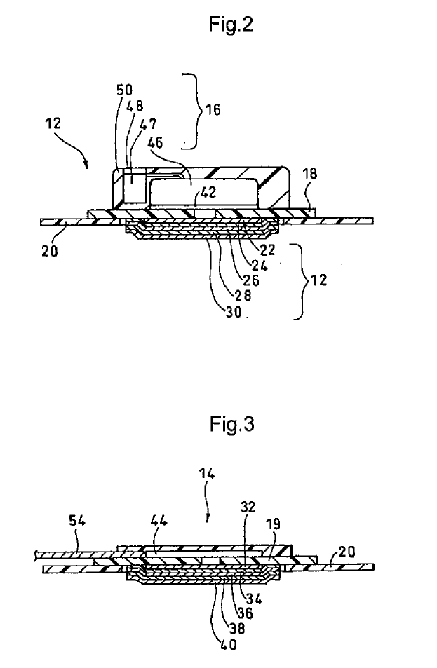

FIG. 2 shows a cross-section of the working electrode assembly 12 of FIG. 1. The working electrode assembly 12 may, in one embodiment, be formed by laminating a working electrode 22, an electrolyte solution holding portion 24, a second ion exchange membrane 26, a drug solution holding portion 28, and a first ion exchange membrane 30 in that order on a lower side of a base sheet 18. These layers may form a circle of 10 to 40 mm in diameter. A circular adhesive sheet 20 may also be arranged on the lower surface of the base sheet 18, so as to substantially surround the working electrode 22.

The base sheet 18 may comprise a variety of materials, including a hard, insulative and elastic resin material, such as a polyethylene terephthalate (PET) resin. The base sheet 18 may be further adapted to help press the working electrode 22 to the first ion exchange membrane 30 against a biological interface of an organism when the adhesive sheet 20 adheres to the biological interface.

The working electrode 22 may include a conductive paint applied to one surface of the base sheet 18 blended with a nonmetal conductive filler, such as a carbon paste. In another embodiment, the working electrode 22 may comprise a copper plate or a metal thin film.

The electrolyte solution holding portion 24 may, in one embodiment, comprise an electrolytic paint applied to the working electrode 22. The electrolytic paint may be any paint containing an electrolyte. Examples of suitable electrolytes include: medical agents (e.g., ascorbic acid (vitamin C) and sodium ascorbate), and organic acids (e.g., lactic acid, oxalic acid, malic acid, succinic acid, and fumaric acid and/or salts thereof). The use of such electrolytes may suppress the generation of oxygen or hydrogen. In addition, blending multiple kinds of electrolytes serving as a combination of buffer electrolyte solutions upon dissolution into a solvent may suppress a change in pH during energization.

The electrolytic paint may be blended with a hydrophilic polymer (e.g., polyvinyl alcohol, polyacrylic acid, polyacrylamide, or polyethylene glycol) in order to facilitate application and improve the film-forming property of the paint. The electrolytic paint may also be blended with an appropriate amount of solvent, such as water, ethanol, or propanol, for adjusting its viscosity. In some embodiments, the paint may be blended with other components, such as a thickener, a thixotropic agent, a defoaming agent, a pigment, a flavor, or a coloring agent.

The second ion exchange membrane 26 may be formed by applying a second ion exchange paint to the electrolyte solution holding portion 24.

The second ion exchange paint may comprise any of a variety of paints containing an ion exchange resin into which an ion exchange group has been introduced, using, for example, an ion having a polarity opposite to that of a drug ion in the drug solution holding portion 28. For example, if a drug whose drug component dissociates to positive drug ions is used in the drug solution holding portion 28, the second ion exchange paint may be blended with an anion exchange resin. On the other hand, if a drug whose drug component dissociates to negative drug ions is used, the second ion exchange paint may be blended with a cation exchange resin.

The drug solution holding portion 28 may comprise a drug paint applied to the second ion exchange membrane 26. The drug paint may contain a drug (including a precursor for the drug) whose drug component dissociates to positive or negative ions (drug ions) as a result of, for example, dissolution into a solvent such as water. Examples of drugs whose drug components dissociate to positive ions include lidocaine hydrochloride as an anesthetic drug and morphine hydrochloride, each as an anesthetic. An example of a drug whose drug component dissociates to negative ions is ascorbic acid as a vitamin agent.

The first ion exchange membrane 30 may comprise a first ion exchange paint applied to the drug solution holding portion 28. The first ion exchange paint may contain an ion exchange resin into which an ion exchange group is introduced, using, for example, an ion having the same polarity as that of the drug ion in the drug solution holding portion 28. Thus, if a drug whose drug component dissociates to positive drug ions is used in the drug solution holding portion 28, the first ion exchange paint may be blended with a cation exchange resin and vice versa.

The above-described ion exchange resins may be obtained by introducing a cation exchange group (i.e., an exchange group using a cation as a counter ion), such as a sulfonic group, a carboxylic group, or a phosphoric group, into a polymer having a three-dimensional network structure, such as a hydrocarbon-based resin (for example, a polystyrene resin or an acrylic resin) or a fluorine-based resin having a perfluorocarbon skeleton.

In another embodiment, the ion exchange resin may be obtained by introducing an anion exchange group (i.e., an exchange group using an anion as a counter ion), such as a primary amino group, a secondary amino group, a tertiary amino group, a quaternary ammonium group, a pyridyl group, an imidazole group, a quaternary pyridinium group, or a quaternary imidazolium group, into a polymer having a three-dimensional network structure, similar to that used to form the cation exchange resin. Of course, different ion exchange resins may be used in other embodiments.

FIG. 3 shows a cross-section of the non-working electrode assembly 14 of FIG. 1. The non-working electrode assembly 14 may, in one embodiment, be formed by laminating a non-working electrode 32, a second electrolyte solution holding portion 34, a third ion exchange membrane 36, a third electrolyte solution holding portion 38, and a fourth ion exchange membrane 40 arranged on a lower side of a non-working base sheet 19 similar to the base sheet 18 in that order. These layers may form substantially the same shape as that of the working electrode assembly 12.

The non-working electrode 32 may be formed similarly to the working electrode 22 in the working electrode assembly 12. In addition, the arrangement and composition of the second electrolyte solution holding portion 34 and the third electrolyte solution holding portion 38 may be the same as or similar to those of the electrolyte solution holding portion 24.

The third ion exchange membrane 36 may comprise an ion exchange paint applied to the second electrolyte solution holding portion 34. The ion exchange paint may be the same as or similar to the first ion exchange paint from which the first ion exchange membrane 30 is formed, and the third ion exchange membrane 36 may function as an ion exchange membrane similar to the first ion exchange membrane 30.

The fourth ion exchange membrane 40 may comprise the same ion exchange paint as that described above with respect to the second ion exchange membrane 26 applied to the third electrolyte solution holding portion 38. The fourth ion exchange membrane 40 may function the same as or similar to the second ion exchange membrane 26.

A working electrode terminal 42 may be arranged on the upper surface of the base sheet 18 opposite the working electrode 22, and conduction may be established between the working electrode terminal 42 and the working electrode 22 of the working electrode assembly 12 through a through-hole arranged on the base sheet 18.

Similarly, a non-working electrode terminal 44 may be arranged on the upper surface of the non-working base sheet 19 opposite the non-working electrode 32, and conduction may be established between the non-working electrode terminal 44 and the non-working electrode 32 of the non-working electrode assembly 14 through a through-hole formed on the non-working base sheet 19.

The DC electric power source 16 may be arranged to cover an upper side of the working electrode terminal 42.

In one embodiment, the DC electric power source 16 comprises a coin battery 46 coupled to current control circuitry 47, which may include a programmable microprocessor or programmable controller. The current control circuitry 47 may include, for example, a one chip integrated circuit operable to programmably control a current that flows to each of the electrode assemblies 12, 14 according to a set pattern. The set pattern may be rewritable from outside based on a signal (e.g., a radio signal) received at a minute antenna 48 (e.g., a loop antenna). A mold resin 50 composed of an insulating material may be used to insulate and integrally mold these electrical components.

The coin battery 46 may be arranged at or near substantially the center of the circular working electrode assembly 12 in a plan view, and the current control circuitry 47 may be placed outside the coin battery 46 and laterally next to the coin battery 46. Each of the coin battery 46 and the current control circuitry 47 may, in one embodiment, be entirely molded within the mold resin into a flat circular shape having an outside diameter substantially equal to that of the circular working electrode assembly 12.

As illustrated in the circuit diagram of FIG. 4, the coin battery 46 may be coupled to the current control circuitry 47, and the cathode side of the circuitry may be connected to the working electrode terminal 42 and the anode side of the circuitry may be connected to the non-working electrode terminal 44 through a lead wire 52.

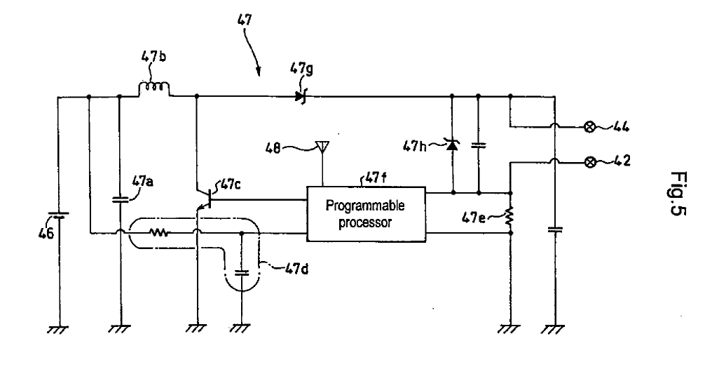

As shown in detail in FIG. 5, the current control circuitry 47 may include: a capacitor 47a for accumulating a charge of the coin battery 46; a coil 47b for collectively discharging the charge accumulated in the capacitor 47a; a switching transistor 47c for turning an output side of the coil 47b on or off; an RC filter 47d; a feedback resistor 47e for detecting a current flowing between the electrode terminals 42, 44 (i.e., through a patient); and a programmable processor 47f operating as a boosting converter for turning the switching transistor 47c on and off to keep a voltage across the feedback resistor 47e at a set value. In FIG. 5, reference symbol 47g denotes a reverse flow preventing diode, and reference symbol 47h denotes a protecting diode. Of course, other analog and digital implementations of the control circuitry 47 may be used in other embodiments.

As shown in FIG. 1, a coupling belt 54 may couple the base sheet 18 and the non-working base sheet 19 and may be formed of a PET film, for example. The lead wire 52 may be arranged to pass through the inside of the coupling belt 54 or may run along a surface of the belt 54.

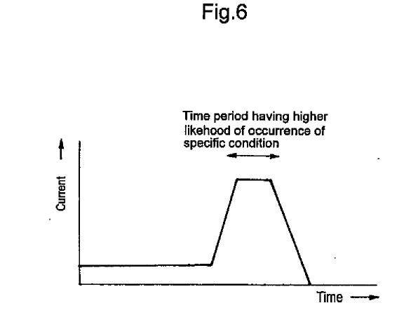

FIG. 6 shows an example of a set pattern for a current flowing to the electrode 12, 14, as controlled by the current control circuitry 47. This set pattern represents an administration pattern for a drug from the iontophoresis device 10. Thus, in one application, a drug may be administered at a predetermined time (e.g., in accordance with an attack at dawn) even though the patch is put in place at another time (e.g., before sleeping at night). Furthermore, even if a patient forgets to peel the patch off the skin, the amount of drug administered may be limited. In one embodiment, a set pattern for the current may be rewritable from outside the iontophoresis device 10 via the antenna 48. In another embodiment, the set pattern may be fixed for each patch, and the antenna 48 may be omitted.

In one embodiment, an electric power source device includes the coin battery 46 and the electric power source circuitry 47 each molded into a flat shape by the mold resin 50 and each having a diameter substantially equal to that of the working electrode assembly 12 in a plan view. As a result, the patch-type working electrode assembly 12 may be formed without a substantial increase in the entire size of the assembly.

In the illustrated embodiment, the DC electric power source 16 is arranged on one side of the working electrode assembly 12. However, in other embodiments, the DC electric power source 16 may be arranged on one side of the non-working electrode assembly 14, or may be arranged on both the working electrode assembly 12 and the non-working electrode assembly 14.

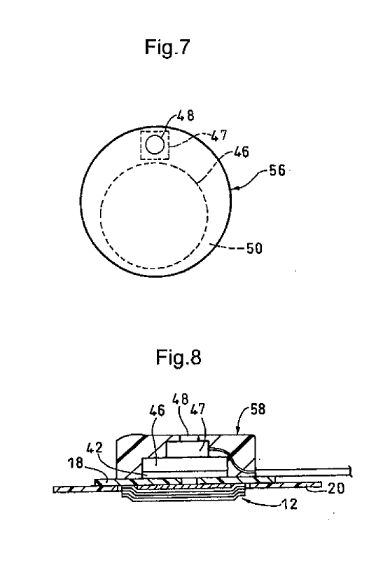

In one embodiment, the coin battery 46 is arranged at the center of the working electrode assembly 12 in a plan view, and the current control circuitry 47 is arranged outside and next to the coin battery 46. However, other embodiments are, of course, possible. For example, as shown in FIG. 7, the coin battery 46 may be shifted from the center of the working electrode assembly 12 in a plan view to one side, and the current control circuitry 47 may be arranged on an opposite side. In this embodiment, an outer diameter of the mold resin 50 may be reduced.

In yet another embodiment, as shown in FIG. 8, the current control circuitry 47 may be arranged and molded on a top side of the coin battery 46. Such an embodiment may be especially useful where a diameter of the working electrode assembly 12 in a plan view is small.

In each of the above embodiments, the iontophoresis device 10 comprises a patch-type working electrode assembly and a non-working electrode assembly. However, other iontophoresis devices may also be used. The DC electric power source is also not limited to a coin battery. In addition, after the current control circuitry 47 has been incorporated, the antenna 48 may be omitted by making the output pattern of the circuitry 47 unrewritable from the outside.

### DESCRIPTION OF REFERENCE NUMERALS

10 IONTOPHORESIS DEVICE
12 WORKING ELECTRODE ASSEMBLY
14 NON-WORKING ELECTRODE ASSEMBLY
16 DC ELECTRIC POWER SOURCE
18 BASE SHEET
19 NON-WORKING BASE SHEET
20 ADHESIVE SHEET
22 WORKING ELECTRODE
24 ELECTROLYTE SOLUTION HOLDING PORTION
26 SECOND ION EXCHANGE MEMBRANE
28 DRUG SOLUTION HOLDING PORTION
30 FIRST ION EXCHANGE MEMBRANE
46 COIN BATTERY
47 CURRENT CONTROL CIRCUITRY
48 ANTENNA
50 MOLD RESIN

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art.

The various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1 An iontophoresis device, comprising:
a working electrode assembly and a non-working electrode assembly for administering a drug by iontophoresis;
an electric power source connected to the working electrode assembly and the non-working electrode assembly; and current control circuitry coupled to the electric power source and operable to programmably control a current that flows to each of the electrode assemblies according to a set pattern;
wherein the set pattern of the current control circuitry is rewritable from outside the iontophoresis device without the contact; and
wherein the drug is released based at least in part on the current.

2. (canceled)

3. The iontophoresis device of claim 1, wherein the working electrode assembly and the non-working electrode assembly are each of a type.

4. The iontophoresis device of claim 1, wherein the working electrode assembly comprises:
a working electrode connected to the electric power source with a same polarity as that of a charged ion of the drug;
an electrolyte solution holding portion holding an electrolyte solution, the electrolyte solution holding portion adjacent a front surface of the working electrode;
a second ion exchange membrane permitting passage of ions having a polarity opposite to that of the charged ion of the drug, the second ion exchange membrane adjacent a front surface of the electrolyte solution holding portion;
a drug solution holding portion holding the drug, the drug solution holding portion adjacent a front surface of the second ion exchange membrane; and
a first ion exchange membrane permitting passage of ions having the same polarity as that of the charged ion of the drug, the first ion exchange membrane adjacent a front surface of the drug solution holding portion; and wherein
the non-working electrode assembly comprises:
the non-working electrode connected to the electric power source having the polarity opposite to that of the charged ion of the drug;
a second electrolyte solution holding portion holding a second electrolyte solution, the second electrolyte solution holding portion adjacent a front surface of the non-working electrode;
a third ion exchange membrane permitting passage of ions having the same polarity as that of the charged ion of the drug, the third ion exchange membrane adjacent a font surface of the second electrolyte solution holding portion;
a third electrolyte solution holding portion holding a third electrolyte solution, the third electrolyte solution holding portion adjacent a front surface of the third ion exchange membrane; and
a fourth ion exchange membrane permitting passage of ions having the polarity opposite to that of the charged ion of the drug, the fourth ion exchange membrane adjacent a front surface of the third electrolyte solution holding portion.

5. An electric power source device for an iontophoresis device having a working electrode assembly and a non-working electrode assembly for administering a drug by iontophoresis, the electric power source device comprising:
a battery;
current control circuitry operable to programmably control a current from the battery to each of the electrode assemblies according to a set pattern; and
an antenna operable to receive a signal used to rewrite the set pattern of the current control circuitry without contact.

6. The electric power source device of claim 5, wherein the current control circuitry is disposed radially outside the battery and side by side next to the battery.

7. The electric power source device of claim 4, wherein the battery is disposed off center from the working electrode assembly, and the current control circuitry is disposed next to the battery on a side of the battery closer to a center of the working electrode assembly.

8. The electric power source device of claim 4, wherein the current control circuitry is disposed on top of the battery.
